(19) United States
(12) Patent Application Publication
(10) Pub. No.: US 2008/0281357 A1
(43) Pub. Date: Nov. 13, 2008

(54) Looped tissue-grasping device

(76) Inventors: An-Min Jason Sung, Warren, NJ (US); Jie Jenny Yuan, Branchburg, NJ (US); Gene W. Kammerer, East Brunswick, NJ (US)

Correspondence Address: GREENBERG TRAURIG, LLP, 200 PARK AVE., P.O. BOX 677, FLORHAM PARK, NJ, 07932, US

(21) Appl. No.: 11/801,414

(22) Filed: May 9, 2007

(51) Int. Cl. A61B 17/04

(52) U.S. Cl. 606/232

(57) ABSTRACT

A looped tissue-grasping device includes a looped suture having two ends swaged to a needle and a plurality of tissue-grasping elements provided on the looped suture. The device is used for joining bodily tissue in surgical applications and wound repair. The configuration of the tissue-grasping elements permit movement of the suture through the tissue in the direction the needle is pulled and, prevent slippage or movement of the suture in a direction opposite to the direction of movement of the needle.

### FIELD OF THE INVENTION

This invention relates generally to tissue-grasping devices, and, more particularly, to such devices which include a looped suture.

### BACKGROUND OF THE INVENTION

Surgical or accidental wounds are typically closed with a length of filament, commonly referred to as a suture, which is introduced into the tissue by a sharp metal needle attached to one thereof. Sutures are used to make stitches to close the wound by holding the tissues together for healing and re-growth. Sutures are used in surgical procedures for wound closure, to close the skin in plastic surgery, to secure damaged or severed tendons, muscles or other internal tissues, and in microsurgery on nerves and blood vessels. Generally, the suture needle is used to penetrate and pass through the tissue, pulling the suture through the tissue. The opposing faces of the tissue are then approximated together, the needle is removed, and the ends of the suture are tied in a knot. The suture forms a loop as the knot is tied. The knotting procedure allows the tension on the filament to be adjusted to accommodate the particular tissue being sutured and to control the approximation, occlusion, attachment or other conditions of the tissue. The ability to control tension is extremely important, regardless of the type of surgical procedure being performed.

Suturing is a time-consuming part of most surgical procedures, particularly in microsurgery and endoscopic surgery, where there is insufficient space to properly manipulate the suture. For adequate closure of some wounds, the suture material must be of a high tensile strength and multiple stitches must be applied. When the tissue structure is weak or when the closure is in a deep layer, the security of the stitch is especially important.

When the wound is long and requires multiple layers of stitches for closure, more time is required to complete the suturing. For example, superficial fascia system (SFS) and deep dermal layer closures during abdominoplasty, body-lifting and body-contouring surgeries are all time-consuming, especially for massive weight-loss patients. The surgeon uses interrupted suturing techniques to close the SFS and deep dermal layers. These techniques contain multiple steps including loading/reloading the needle, penetrating the tissue with the needle and advancing it through the tissue, knotting, and cutting the suture. Applying a continuous or running stitch can reduce this time. However, the continuous stitching requires constant tensioning during suturing to maintain the proper wound approximation, and may not be as secure as the interrupted stitch, because if one portion of the suture fails, then the whole wound opens. In contrast, when interrupted stitches are used, if one stitch fails the others are not affected. Thus, for deep closures of thick tissues, multiple interrupted sutures are used instead of a running stitch. If too much tension is applied, the tightened suture loop of the interrupted stitch may also constrict blood flow to the tissue it surrounds, promoting necrosis of the wound margins, which may compromise healing and increase infection risks.

The knots which secure the sutures in tissue also present problems. For instance, the tissue is distorted when it is secured by the suture under excess tension from the knots. Localized tensions from the knots also contribute to scar formation. The bulk of the knots is also an impediment to wound healing in internal applications. Additionally, the bulk of the knot may be detectable or palpable by the patient through the layers of tissue. For permanent sutures, such as those made from polyesters or polypropylenes, these knots remain indefinitely. For absorbable sutures, such as those made from polydioxanone or polyglactin, the knots eventually disappear when the suture material is absorbed. However, while the knots are present, and in some cases for an extended period of time after they are gone, the area can still remain sensitive and/or impacted by their previous presence. Consequently, minimizing the knot mass and size, as well as position, is important to the comfort of the patient, while maintaining the security of the closure. Knots are also believed to be the major source of surgical site infection, as they have the potential to hold bacteria during surgical procedures.

Alternatives to conventional sutures for wound closure are known, including fasteners such as staples, clips, tacks, clamps and the like. The fasteners are usually positioned transversely across a wound for joining or approximating each side of adjacent tissue layers laterally. Fasteners have relatively high strength and save time, but are not as accurate as sutures and are bulky and may be painful to remove. Fasteners are also generally unsuitable for deeper layers of tissue. Moreover, fasteners do not provide the advantage of adjustable tension obtained by the knotting of a length of suture material.

Surface adhesive tapes and glues are often used on skin to hold small wounds closed to permit healing. However, these products have relatively low tensile strength and are not useful in many situations which require high holding forces. Other proposed techniques include electrical coagulation and lasers. However, no acceptable alternative has been found which offers the advantages of suturing and tying in most surgical procedures.

One possible alternative to tying knots is the use of a barbed suture. A barbed suture includes an elongated body having one or more spaced barbs projecting from the surface of the body along the length of the body. The barbs are configured to allow passage of the suture in one direction through tissue, but resist movement of the suture relative to the tissue in the opposite direction. In wound closure, a barbed suture is passed through tissue at each of the opposed sides of a wound. The wound is closed by pushing the sides of the wound together with the barbs, maintaining the sutures in place and resisting movement of the tissue away from this position. The advantage of using barbed sutures is the ability to introduce tension in the tissue with less slippage of the suture in the wound. The barbed suture spreads out the holding forces evenly, thereby significantly reducing tissue distortion. The tension caused by placing the suture in the tissue is directed along the length of the suture in both directions. A unidirectional barbed suture will only hold in one direction, and so knots are required at one end (i.e., the end towards which the barbs face) to keep it secure. This end is usually the end where the suturing is started on an incision line. This defeats some of the advantage of the barbed suture over the plain suture. A bidirectional barbed suture can overcome this disadvantage because the barbs extend in both directions. However, this means the bidirectional barbed suture needs to be passed through the tissue in two opposing directions. This has been achieved by double-arming the suture with a needle at both ends. While double-armed sutures are known and used in surgery, the technique for applying such sutures differs significantly from that for applying single-armed sutures, necessitating additional training and skill development by surgeons to use double-armed sutures. Since they are used infrequently, double-armed sutures are generally applied less efficiently than traditional single-armed sutures. Moreover, the use of a double-armed suture makes surgical suturing more complicated and inconvenient for the surgeon. For instance, when using two needles on opposite sides of the suture, the surgeon starts the stitching in the middle of the suture, proceeds on one side, and then continues on the other side. The double-armed suture also requires the surgeon to move from one side of the surgical setting, and patient, to the other side to complete loops with both needles. Also, double-arming the suture makes it difficult for the surgeon to estimate suturing lengths, and sets a specific length for the proximal barbed section, limiting its usefulness. In addition, while bi-directional barbed sutures have the potential to eliminate knots, their strength is reduced when the suture is cut to form barbs thereon.

The prior art also realizes the benefits of looped sutures. However, like regular sutures without loops, looped sutures suffer from shortcomings (e.g., surgeons still need to anchor the end of the suture, and tension must be applied to the suture by another person while the surgeon makes successive stitches).

For the foregoing reasons, there is a need for a wound closure device for joining tissue in surgical applications and wound repair which is efficient, expedites the surgical procedure and minimizes the mass and size of material used to make both the proximal anchoring and the distal anchoring of the suture. There is also a need to develop a suture device that maintains the strength of the suture, and at the same time eliminates knots. Ideally, the new device allows a surgeon to suture in an efficient manner to quickly approximate the tissue with appropriate tension and security and with minimal material. In use, the new device could preserve blood flow, improve wound healing strength, prevent distortion of the tissue and minimize scarring. Furthermore, the new device could be used in various types of tissues, such as for closing wounds of friable tissue without resulting in a cheese wire effect, and in connection with methods which incorporate the self-retaining benefits of the barbed suture with the holding power of conventional suturing methods (e.g., the device could be utilized in surgical applications where space is limited and knot-tying is restricted or made more difficult, such as in microsurgery, endoscopic or arthroscopic surgery).

### SUMMARY

A tissue-grasping device includes a needle and a looped suture attached to the needle. The looped suture has a closed end opposite the needle and first and second strands extending between the closed end and the needle. At least one of the first and second strands includes one or more tissue-grasping elements provided thereon. For instance, tissue-grasping elements may be provided on inner, outer, or both inner and outer lateral surfaces of the first and second strands.

The closed end of the tissue-grasping device is not provided with any tissue-grasping elements. The first strand includes a first portion proximate the needle and a second portion proximate the closed end. Both the first and second portions of the first strand do not have tissue-grasping elements thereon. Likewise, the second strand has a first portion proximate the needle and a second portion proximate the closed end, neither of which has tissue-grasping elements thereon.

The tissue-grasping elements each include a leading edge proximate to the needle and a trailing edge distal to the needle. The leading and trailing edges have a concaved shape where they merge with the looped suture. In one embodiment, the concaved shape of the trailing edge forms a recess which extends laterally into the looped suture. The tissue-grasping elements are substantially shark fin-shaped in another embodiment. Yet another embodiment has a trailing edge which includes a plurality of serrations thereon.

These and other features and advantages of the present invention will become apparent from the following more detailed description, when taken in conjunction with the accompanying drawings which illustrate, by way of example, the principles of the invention.

### BRIEF DESCRIPTION OF THE DRAWINGS

For a better understanding of the present invention, reference is made to the following detailed description of various exemplary embodiments considered in conjunction with the accompanying drawings, in which:

FIG. 1 is a schematic illustration of an exemplary embodiment of a looped tissue-grasping device constructed in accordance with the present invention;

FIG. 2 is a schematic illustration of a second exemplary embodiment of a looped tissue-grasping device constructed in accordance with the present invention;

FIG. 3 is a schematic illustration of a third exemplary embodiment of a looped tissue-grasping device constructed in accordance with the present invention;

FIG. 4 is s perspective view of a fourth exemplary embodiment of a looped tissue-grasping device constructed in accordance with the present invention;

FIG. 5 is a top plan view of the looped tissue-grasping device illustrated in FIG. 4;

FIG. 6 is a cross-sectional view, taken along section line 6-6 and looking in the direction of the arrows, of the tissue-grasping device illustrated in FIG. 5;

FIG. 7 is a cross-sectional view of a fifth exemplary embodiment of a looped tissue-grasping device constructed in accordance with the present invention, which is similar to the cross-sectional view of FIG. 6;

FIG. 8 is a partial top plan view of a sixth exemplary embodiment of a looped tissue-grasping device constructed in accordance with the present invention;

FIG. 9 is a partial top plan view of a seventh exemplary embodiment of a looped tissue-grasping device constructed in accordance with the present invention;

FIG. 10A is a schematic illustration of an eighth exemplary embodiment of a looped tissue-grasping device constructed in accordance with the present invention;

FIG. 10B is a cross-sectional view taken along section line B-B and looking in the direction of the arrows, of the looped tissue-grasping device illustrated in FIG. 10A;

FIGS. 11A-11C are schematic illustrations of the looped tissue-grasping device illustrated in FIGS. 10A and 10B, including needles having different sizes and cross-sectional areas;

FIG. 12 is a partial schematic illustration of the looped tissue-grasping device illustrated in FIG. 1, wherein the needle has been replaced with a different needle; and

FIGS. 13A-D are schematic illustrations which show the looped tissue-grasping device of the present invention in use.

### DETAILED DESCRIPTION

Before explaining the present invention in detail, it should be noted that the invention is not limited in its application or use to the details of construction and arrangement of parts illustrated in the accompanying drawings and description. The invention as illustrated may be implemented or incorporated in other embodiments, variations and modifications, and may be practiced or carried out in various ways.

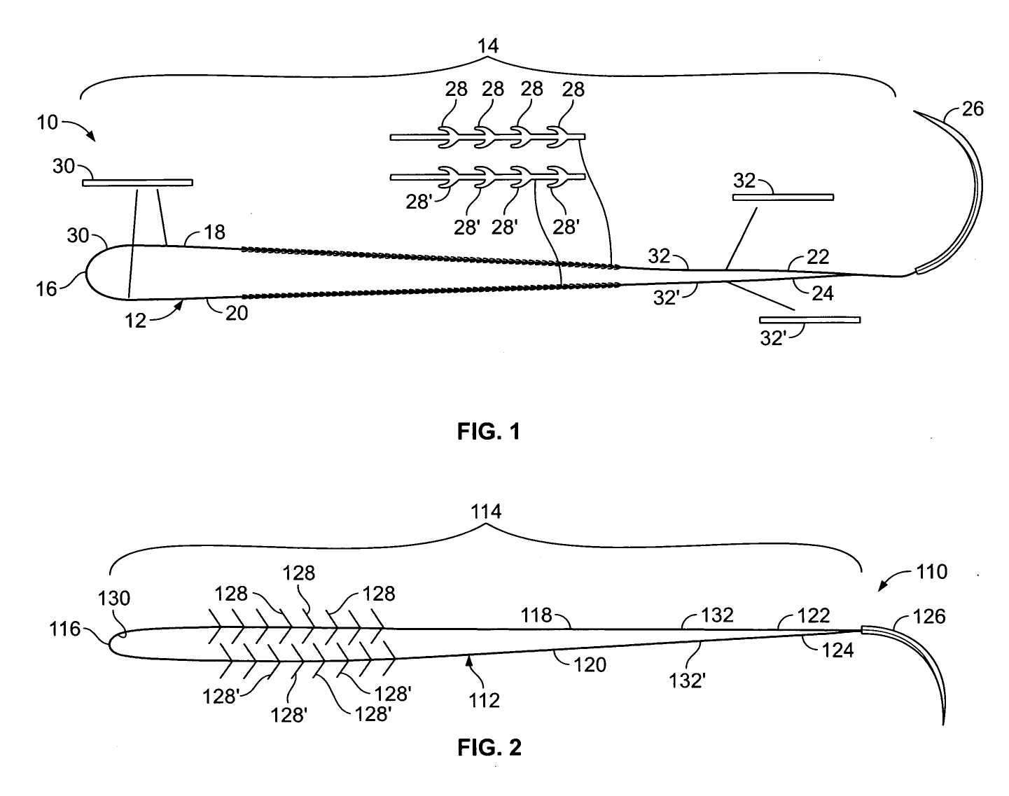

With reference to FIG. 1, an exemplary embodiment of the looped tissue-grasping device 10 of the present invention is illustrated as having an elongated body 12 made from a suitable polymeric material in the form of a monofilament, a co-extruded monofilament, a braided monofilament, a ribbon, or multi-filaments configured into a braid or a substantially flat structure such as a braided or woven ribbon. Suitable polymeric materials include absorbable materials such as polydioxanone, polyglactin, polyglycolic acid, copolymers of glycolide and lactide, polyoxaesters and poly caprolactone, as well as non-absorbable materials such as polypropylene, polyethylene, polyvinylidene fluoride (PVDF), ultra high molecular weight polyethylene (UHMWPE), polyesters, polyethylene terephthalate, glycol-modified polyethylene terephthalate, polytetrafluoroethylene, fluoropolymers, nylons and the like, and combinations thereof, including combinations of absorbable and non-absorbable materials. Preferable materials include, but are not limited to, polypropylene, UHMWPE, polydioxanone, and copolymers of poly (glycolide-co-caprolactone).

Still referring to FIG. 1, the elongated body 12 is arranged to form a looped suture 14 having a closed end 16 and two legs, or strands 18, 20, which extend in a substantially axial direction away from the closed end 16 and terminate in free ends 22, 24, respectively. Each of the strands 18, 20 has an outer lateral surface distal to the other strand, and an inner lateral surface proximate to the other strand. The free ends 22, 24 of the strands 18, 20 are secured together and affixed to a needle 26.

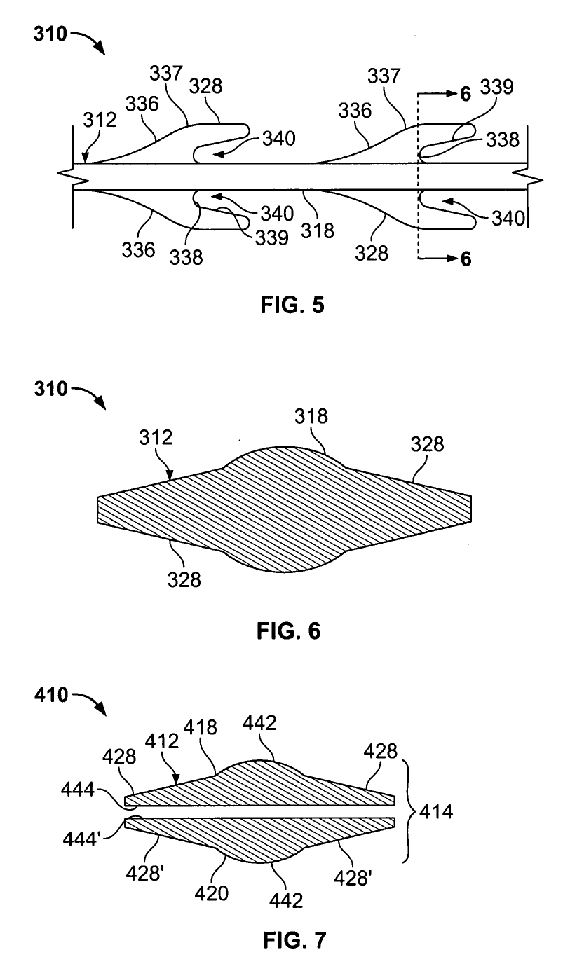

The strand 18 includes a plurality of tissue-grasping elements 28, while the strand 20 includes a plurality of tissue-grasping elements 28′. The term “tissue-grasping elements” is defined herein to include protrusions, barbs and other projections, but is not restricted to such structures. The tissue-grasping elements 28, 28′ are oriented on the elongated body 12 so as to permit movement of the looped suture 14 through the tissue in the same direction as the needle 26 being passed through the tissue, and to prevent slippage or movement of the looped suture 14 in a direction opposite to the movement of the needle 26. More particularly, the tissue-grasping elements 28, 28′ extend in a direction such that their free ends are proximal to the closed end 16. Furthermore, where the elongated body 12, and the strands 18, 20, extend in a substantially axial direction, the tissue-grasping elements 28 extend laterally, from one or both of the lateral surfaces of the strand 18, and the tissue-grasping elements 28′ extend laterally, from one or both of the lateral surfaces of the strand 20. The tissue-grasping elements 28, 28′ each include a leading edge proximate to the needle 26 and a trailing edge distal to the needle 26, as explained further hereinbelow in connection with an alternate embodiment of the present invention which is illustrated in FIG. 5.

The tissue-grasping device 10 may be manufactured in the following manner to form the tissue-grasping elements 28, 28′. The elongated body 12 is formed from an appropriate quantity of a polymeric feedstock material (such as one of the above-listed materials), and is placed in the bottom half of a mold. The elongated body 12 is pressed by the top half of the mold, which has the shape of the tissue-grasping elements 28 cut into it. The tissue-grasping elements 28 are thereby formed on the elongated body 12 and the excess material is discarded. This step produces the strand 18 with the tissue-grasping elements 28 formed thereon. The top half of the mold is then rotated 180 degrees so that the tissue-grasping element cut-outs are facing the opposite direction, and the remaining portion of the elongated body 12 is pressed in the mold. This produces the strand 20 with its tissue-grasping elements 28′ formed thereon and facing the opposite direction from the tissue-grasping elements 28.

Until this point, the elongated body 12 has not been folded, so that the strands 18 and 20 are collinear, with the central clear section 30 between them. A crease is then introduced in the elongated body 12 within the central clear region 30. The elongated body 12 is then folded at the crease to form a looped suture 14 with the strands 18, 20 having substantially equal lengths and tissue-grasping elements 28, 28′ which are aligned with respect to each other. The free ends 22, 24 of the strands 18, 20 are then secured together before being swaged into the needle 26.

The elongated body 12 may also be formed with tissue-grasping elements 28, 28′ using other methods known in the prior art, such as injection-molding, insert injection-molding, co-injection-molding, stamping, chemical etching, progressive die cutting (e.g., using a rotary die), and laser cutting. Alternatively, the tissue-grasping elements 28, 28′ may also be formed by making cuts into and along the elongated body 12 (see FIG. 1A).

With continued reference to FIG. 1, a central clear region 30 having no tissue-grasping elements is provided between the strands 18, 20 of the elongated body 12 and distal to the needle 26 so as to include the closed end 16 of the looped suture 14. The central clear region 30 is so positioned because the presence of tissue-grasping elements at the closed end 16 would interfere with the passage of the needle 26 and the strands 18, 20 through the loop formed by the closed end 16 in forming stitches in tissue (see FIGS. 13A-D). The central clear region 30 is made as short as possible, so as to maximize the holding strength of the looped suture 14 (by providing an optimal number of tissue-grasping elements 28, 28′ thereon), and to prevent the looped suture 14 from loosening and relaxing after it is stitched into the tissue. On the other hand, the central clear region 30 must be long enough to prevent or minimize the rubbing together of sections of the strands 18, 20 having tissue-grasping elements 28, 28′, respectively. The strands 18, 20 rubbing together may damage the tissue-grasping elements 28, 28′ thereon, and result in the premature failure of the looped suture 14, especially when the tissue-grasping elements 28, 28′ are formed by cutting into and along the elongated body 12. The length of the center clear region 30 ranges from 0.5 to 8 inches, and is preferably between 1 and 6 inches.

FIG. 1 also illustrates terminal clear regions 32, 32′ having no tissue-grasping elements 28, 28′ provided along the free ends 22, 24 of the strands 18, 20, respectively, so as to be proximate the needle 26. The terminal clear regions 32, 32′ are free of tissue-grasping elements so that the surgeon can easily grasp the portion of the looped suture 14 proximate the needle 26 when stitching the tissue. The length of the terminal clear regions 32, 32′ ranges from 1 to 6 inches, is preferably between 2 and 5 inches, and is most preferably between 3 and 4 inches.

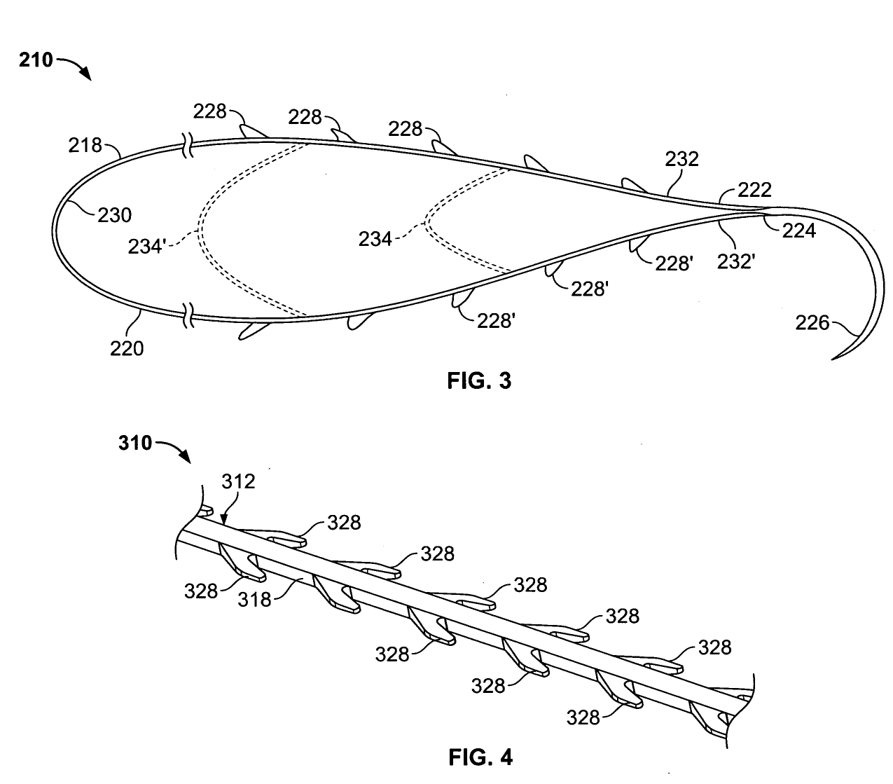

In general, the tissue drag which occurs during stitching depends on the overall size of the suture. For the tissue-grasping device 10, the size of its cross-sectional area is a good indication of its overall size. When the tissue-grasping elements 28, 28′ are arranged on the respective strands 18, 20 so as to be substantially aligned, or “across from” each other (see FIGS. 1 and 3), the cross-sectional area of the tissue-grasping device 10 is larger than if the tissue-grasping elements are “offset” with respect to each other, such that only one set of tissue-grasping elements 28 (or 28′) is present at any given location along the length of the looped suture 14 (see FIG. 2).

Before discussing FIG. 2, it should be noted that the elements of FIG. 2 which correspond to elements described above in connection with the embodiment of FIG. 1 will be designated by corresponding reference numerals increased by one hundred. Unless otherwise specified, the alternate embodiment of FIG. 2 is constructed and operates in the same manner as the embodiment of FIG. 1.

Referring now to FIG. 2, there is shown a looped tissue-grasping device designated by reference number 110. The looped tissue-grasping device 110 differs from that of the first embodiment illustrated in FIG. 1 in that it is provided with tissue-grasping elements 128 on the strand 118 which are offset relative to the closest tissue-grasping elements 128′ on the strand 120. The offset distance ranges from 1 to 1,000 mil, and preferably, it is between 10 and 100 mil.

Still referring to FIG. 2, the offset arrangement of the tissue-grasping elements 128, 128′ provides better tissue engagement, thereby increasing the holding strength of the tissue-grasping elements 128, 128′. This is because, when the tissue-grasping elements 128, 128′ are offset, there is less or no interference among them as the looped suture 114 is being placed in the tissue. The reduction or elimination of interference among the tissue-grasping elements 128, 128′ also reduces the potential for damage to the tissue-grasping elements 128, 128′ and/or the looped suture 114. Any such damage would tend to reduce the holding strength and the tensile strength of the tissue-grasping elements 128,128′ and the looped suture 114.

The tissue-grasping device 110 may be manufactured using the method described hereinabove, with the following modifications. After the crease is introduced in the central clear region 130, the elongated body 112 is folded at the crease to form a looped suture 114 with the strands 118, 120 having unequal lengths. The strands 118, 120 are then arranged so that their tissue-grasping elements 128, 128′ are offset with respect to each other. The free end of the longer strand is then trimmed so as to have a length substantially equal to that of the shorter strand. The free ends 122, 124 of the strands 118, 120 are then secured together and swaged into the needle 126.

Reference is now made to FIG. 3, which illustrates another alternate embodiment of the looped tissue-grasping device of the present invention. In describing this alternate embodiment, elements which correspond to elements described above in connection with the embodiment of FIG. 1 will be designated by corresponding reference numerals increased by two hundred. Unless otherwise specified, the alternate embodiment of FIG. 3 is constructed and operates in the same manner as the embodiment of FIG. 1.

The looped tissue-grasping device illustrated in FIG. 3 is designated by reference number 210, and differs from the embodiments of FIGS. 1 and 2 in that tissue-grasping elements 228, 228′ are provided on only one of the lateral surfaces of each of the strands 218, 220, respectively. Preferably, the tissue-grasping elements 228, 228′ are provided on the outer lateral surface of each of the strands 218, 220, respectively, as shown in FIG. 3. Other configurations of the tissue-grasping elements on only one lateral surface of each strand are also possible. For example, tissue-grasping elements may be provided on the inner lateral surface of each of the strands.

The arrangement of the tissue-grasping elements 228, 228′ on one lateral surface of each of the strands 218, 220 is advantageous, especially when the tissue-grasping elements 228, 228′ are formed along the elongated body 212. For example, in a monofilament with a cross section having multiple sides and apexes (e.g., a triangular cross section, discussed hereinbelow, in connection with FIGS. 10A and 10B), the tissue-grasping elements 228, 228′ may be formed on one of the apexes of each strand 218, 220 instead of on all of the apexes. When the tissue-grasping elements 228, 228′ are formed on one of the apexes, fewer cuts are made into the cross-sectional area of the elongated body 212 (and therefore the looped suture 214), which enables the suture 214 to retain a higher tensile strength than sutures having tissue-grasping elements cut into multiple apexes. Under similar tensile loading conditions, the looped suture 214 with the tissue-grasping elements 228, 228′ formed on one apex allows the formation of larger tissue-grasping elements 228, 228′, which are preferred for deeper tissue penetration in certain types of tissues and procedures. For example, in closing fatty tissues, a looped suture having larger tissue-grasping elements is expected to provide better tissue engagement and holding strength than looped sutures with small tissue-grasping elements.

Like all of the embodiments described above, the strands 218, 220 of the tissue-grasping device 210 form a single closed loop which acts as the initial anchor to counteract the holding force of the tissue-grasping elements 228, 228′. However, the looped suture 214 loses its functionality after being cut away from the needle 226. Surgeons have attempted to overcome this limitation by tying the two free ends of the suture together to reform the loop. However, this technique tends to result in damage to the tissue-grasping elements. To remedy this situation, the tissue-grasping device 210 may be modified to provide more than one loop. Additional loops enable the surgeon to start a new line of stitching after a running stitch is completed, but before the wound is completely closed, whereby the multi-looped device may be used multiple times at different locations of the incision because the multiple loops act as multiple anchors for holding the suture and stopping it from slipping forward.

In order to provide the tissue-grasping device 210 with multiple loops, one or more supplemental strands of suture can be added between the two strands 218, 220 to form, for example, additional loops 234, 234′ (shown in phantom in FIG. 3). As discussed in the preceding paragraph, the additional loops 234, 234′ enable the surgeon to use the multi-looped tissue-grasping device 210 as multiple suturing units. More particularly, the additional loops 234, 234′ may be used to anchor new stitches after the initial line of stitches has been placed.

The additional loops 234, 234′ may be formed by adhering the supplemental suture strands to the strands 218, 220 using glue, adhesive, sonic welding, laser welding, heat pressure, injection-molding, insert-molding or any other techniques to secure the supplemental strands on the strands 218, 220. A multi-looped tissue-grasping device may also be formed by swaging supplemental strands into the needle, so that each additional loop is independent of the other loops. Another way to form a multi-looped tissue-grasping device is to split the strands 218, 220 of the looped suture 214 along their lengths and then swag the split-off portions of the strands 218, 220 into the needle 226 to form multiple loops 234, 234′.

The number of supplemental strands of suture may be modified to include as many loops as desired. The length of the supplemental strands may also be modified to provide loops of various lengths, depending on the needs of the surgeon and the nature of their use. Additional loops may also be formed in the first and second embodiments of the present invention illustrated in FIGS. 1 and 2, respectively, and in other embodiments of the present invention.

One of the strands of the looped suture may be provided with fewer or no tissue-grasping elements. For example, the tissue-grasping elements may be provided on only one strand, whereby the looped suture will include a first strand having higher tensile strength (i.e., the strand without tissue-grasping elements) and a second strand having higher tissue-holding strength (i.e., the strand with tissue-grasping elements). The resulting “combination” looped suture is therefore expected to have better overall wound-holding strength.

In order for the strand with higher tensile strength to absorb more of the tensile forces generated during suturing, the strands of the looped suture are formed in a twisted or helical configuration. More particularly, in one embodiment, the strand with higher tensile strength having no tissue-grasping elements is substantially linear (i.e., straight) and acts as a “core,” while the strand with more tissue-grasping elements is twisted around the core during the manufacturing process.

Whereas both strands of the looped suture having this twisted configuration are stretched when subjected to tensile forces, the straight strand acting as the core carries most of the tensile load. In the meantime, the twisted strand begins to rotate in response to the tension, thereby “untwisting” and straightening itself. As a result, the twisted strand is subjected to less material deformation, which helps to maintain the structure and functionality of the tissue-grasping elements formed thereon.

In addition to the preferred tensile loading distribution in the strands, this twisted configuration of the looped suture also eliminates the potential separation of the strands. In other words, the twisted configuration keeps the two strands aligned and packed together. Other embodiments of the twisted configuration are also possible.

Turning now to the tissue-grasping elements, their physical characteristics (i.e., size, shape, etc.) and overall design play a key role in determining the performance of the device of the present invention. Specifically, the physical characteristics and design of the tissue-grasping elements affect many factors relating to the suturing process, including insertion forces, tissue drag, bending resistance, tissue engagement and holding strength of the tissue-grasping elements, and the tensile strength of the suture.

While several methods for forming tissue-grasping elements are discussed in the prior art, the preferred manufacturing methods of profile-punching and press-forming the looped sutures, as described previously, allow the tissue-grasping elements to be formed to include a variety of design features, as illustrated in FIGS. 4-9. While these figures illustrate embodiments having different modified tissue-grasping elements, alternate embodiments and modifications are also possible.

Reference is now made to FIGS. 4, 5 and 6, which illustrate one such alternate embodiment of the looped tissue-grasping device of the present invention. In describing this alternate embodiment, elements which correspond to elements described above in connection with the embodiment of FIG. 1 will be designated by corresponding reference numerals increased by three hundred. Unless otherwise specified, the alternate embodiment of FIGS. 4, 5 and 6 is constructed and operates in the same manner as the embodiment of FIG. 1.

Referring still to FIGS. 4, 5 and 6, a looped tissue-grasping device 310 includes an elongated body 312 from which a loop having two strands is formed. For the sake of clarity, only the strand 318 is shown in FIGS. 4, 5 and 6, with the understanding that the strand 320, while not shown, is constructed and operates in the same manner as the strand 318. Tissue-grasping elements 328 are formed on the elongated body 312 in a “shark fin” shape. More particularly, as illustrated in FIG. 5, each of the tissue-grasping elements 328 includes a gradually-sloping fillet 336 on a leading edge 337 thereof and an enlarged (i.e., widened) radius 338 on a trailing edge 339 thereof. The radius 338 is curved so as to reduce stress concentration. An elongated space 340 is provided between each of the trailing edges 339 and the strand 318.

The fillet 336 provides the tissue-grasping elements 328 with a low profile, which reduces the insertion forces exerted on the tissue-grasping elements 328 during suturing. The tissue-grasping elements 328 also maintain high tissue-holding strength by capturing a large volume of tissue in the elongated space 340. The fillet 336 includes additional material, which increases the bending resistance of the tissue-grasping elements 328.

Reference is now made to FIG. 7, which illustrates another embodiment of the present invention. In describing this alternate embodiment, elements which correspond to elements described above in connection with the embodiment of FIG. 1 will be designated by corresponding reference numerals increased by four hundred. Unless otherwise specified, the alternate embodiment of FIG. 7 is constructed and operates in the same manner as the embodiment of FIG. 1.

With continued reference to FIG. 7, a looped tissue-grasping device 410 includes an elongated body 412 from which a loop 414 having two strands 418, 420 is formed. Tissue-grasping elements 428, 428′ are formed on the elongated body 412 in a shark fin shape, similar to those of the embodiment of the present invention illustrated in FIGS. 4, 5 and 6. However, the cross-sectional shape of the elongated body 412 (and the looped suture 414 and strands 418, 420) differs from the previously-described embodiment. More particularly, the elongated body 412 is formed to have a substantially flat surface, such that the strands 418, 420 of the looped suture 414 will each have a substantially flat inner lateral surface as well. As illustrated in FIG. 7, the strand 418 has a rounded outer lateral surface 442 and a substantially flat inner lateral surface 444 opposite thereto. The strand 420 is similarly shaped with a rounded outer lateral surface 442′ and a substantially flat inner lateral surface 444′.

Referring still to FIG. 7, when the looped suture 414 is formed, the flat inner lateral surfaces 444, 444′ of the strands 418, 420 are positioned to face each other. This arrangement aligns the two strands 418, 420, which facilitates the swaging of the strands 418, 420 into the needle (not shown), insertion of the needle into tissue, and suture engagement in tissue. Alternative cross-sectional shapes are possible.

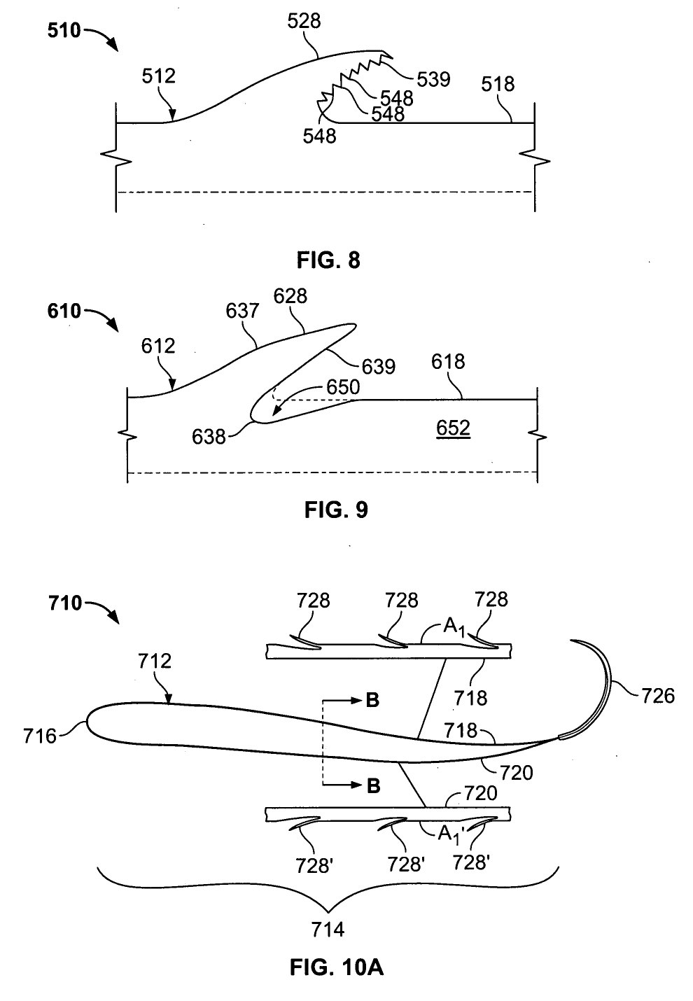

Reference is now made to FIG. 8, which illustrates another embodiment of the present invention having modified tissue-grasping elements. In describing this alternate embodiment, elements which correspond to elements described above in connection with the embodiment of FIG. 1 will be designated by corresponding reference numerals increased by five hundred. Unless otherwise specified, the alternate embodiment of FIG. 8 is constructed and operates in the same manner as the embodiment of FIG. 1.

In addition to the aforementioned features of the shark fin-shaped tissue-grasping elements, a trailing edge 539 of the tissue-grasping elements 528 may be formed so as to enhance its tissue-grasping properties. More particularly, a plurality of serrations 548 is provided on the trailing edge 539 of each of the tissue-grasping elements 528, as illustrated in FIG. 8. The serrations 548 enhance the tissue-grasping ability of the tissue-grasping elements 528 without affecting other major suture performance and tissue-holding strength. The serrations 548 may be formed in a variety of shapes and sizes.

Another problem associated with prior art sutures is the mechanical failure of the tissue-grasping elements at their base, which occurs in response to the high bending stresses exerted on the tissue-grasping elements during the stitching of the suture into the tissues. This shear stress failure mechanism is especially dangerous for sutures made from materials which are extruded or otherwise manufactured in a way that aligns the molecules of the material in parallel linear paths along the length of the elongated body. The alignment associated with these manufacturing processes makes the outwardly-extending tissue-grasping elements more susceptible to mechanical failure where the linear path of the material is interrupted (i.e., proximate the radii of the tissue-grasping elements, where the radii form an intersection point with the strand). This intersection point is known as a “hot spot.” For the reasons explained above, the tissue-grasping elements tend to fail at the hot spot in response to high bending stresses.

Reference is now made to FIG. 9, which illustrates another embodiment of the present invention having modified tissue-grasping elements. In describing this alternate embodiment, elements which correspond to elements described above in connection with the embodiment of FIG. 1 will be designated by corresponding reference numerals increased by six hundred. Unless otherwise specified, the alternate embodiment of FIG. 9 is constructed and operates in the same manner as the embodiment of FIG. 1.

A looped tissue-grasping device 610 according to the present invention is illustrated in FIG. 9 as modified to avoid the aforementioned mechanical failures. More particularly, the trailing edge 639 forms a recess 650 in the elongated body 612 proximate the radius 638 of each of the tissue-grasping elements 628. The recess 650 redistributes the large bending stresses from the radius 638 of the tissue-grasping element 628 to a more massive core 652 of the elongated body 612, which includes a larger region of material to absorb the high bending stresses. The recess 650 thereby moves the hot spot into a more mechanically stable part of the device 610. The inclusion of the recess 650 therefore reduces the incidence of mechanical failure experienced by the tissue-grasping elements 628 as a result of the high bending stresses.

Now referring to FIGS. 10A and 10B, another embodiment of the tissue-grasping elements of the present invention is illustrated. In describing this alternate embodiment, elements which correspond to elements described above in connection with the embodiment of FIG. 1 will be designated by corresponding reference numerals increased by seven hundred. Unless otherwise specified, the alternate embodiment of FIGS. 10A and 10B is constructed and operates in the same manner as the embodiment of FIG. 1.

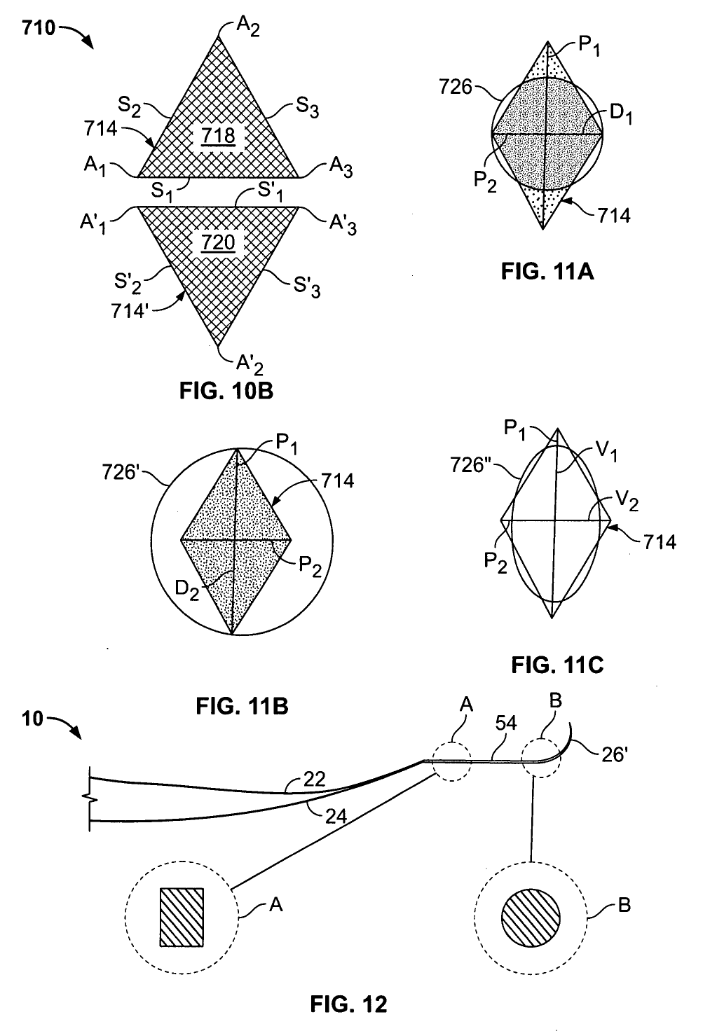

As explained previously, the tissue-grasping elements 728, 728′ may be cut into and along the elongated member 712, as illustrated in FIG. 10A. More particularly, the enlarged, detailed sections of FIG. 10A show that the elongated body 712 is sliced at intervals along strands 718, 720 to form the tissue-grasping elements 728, 728′, respectively. FIG. 10B illustrates the triangular-shaped cross sections of strands 718, 720 at a location between (i.e., without) tissue-grasping elements 728, 728′. Each of the strands 718, 720 is provided with three apexes A₁, A₂, A₃ and A₁′, A₂′, A₃′, respectively, as well as three sides S₁, S₂, S₃ and S₁′, S₂′, S₃′, respectively. When corresponding sides S₁ and S₁′ of the strands 718, 720 are placed in proximity to each other (as in the area where the looped suture 714 attaches to the needle 726), the respective triangular-shaped cross-sectional areas of the strands 718, 720 cooperate such that the resulting suture loop 714 has a parallelogram-shaped cross section (i.e., diamond shaped). For the sake of clarity, the sides S₁ and S₁′ of the strands 718, 720 are illustrated as being spaced apart. However, the strands 718, 720 are arranged, at least in the area where the looped suture 714 attaches to the needle 726, such that the sides S₁ and S₁′ engage each other (see FIGS. 11A-11C).

The triangular-shaped cross section of the elongated body 712 is ideal for use in the cutting method of forming the tissue-grasping elements 728, 728′, since the tissue-grasping elements 728, 728′ may be cut along one, two or all three of the apexes. Referring again to FIGS. 10A and 10B, the tissue-grasping elements 728, 728′ have been cut in only one of the apexes of the elongated body 712, thereby providing tissue-grasping elements 728 along the apex A₂ of the strand 718 and tissue-grasping elements 728′ along the apex A₂′ of the strand 720. As further illustrated in FIG. 10B, the apexes A₂, A₂′ in which the tissue-grasping elements 728, 728′ are formed are the “outward-facing” apexes of the suture 714. In the meantime, the sides S₁ and S₁′ of the triangular-shaped cross sections of the respective strands 718, 720 are arranged proximate to each other, as described previously. The parallelogram-shaped cross section of the suture 714 facilitates improved tissue engagement of the tissue-grasping elements 728, 728′ on strands 718, 720, respectively.

As discussed above, to form the looped tissue-grasping devices of the various embodiments of the present invention, the two strands of the looped suture are swaged into the same needle so that they pass through the same needle hole in the tissue during wound closure. Referring again FIG. 10B, the tissue-holding strength of the looped tissue-grasping device 710 may be enhanced by including a needle having a cross-sectional area that is shaped so as to be similar to the parallelogram-shaped cross-sectional area of the suture 714.

Referring now to FIG. 11 A, a needle 726 is illustrated as having a circular-shaped cross-sectional area which is smaller than the parallelogram-shaped cross-sectional area of the suture 714. More particularly, the parallelogram-shaped cross-sectional area of the suture 714 has a major axis P₁ and a minor axis P₂, wherein the minor axis P₂ is approximately equal to the diameter D₁ of the circular-shaped cross section of the needle 726. This formation results in a greater dragging force during insertion, such that the needle 726 may retain higher holding forces.

Another needle 726′ is shown in FIG. 11B having a circular-shaped cross-sectional area which is larger than the parallelogram-shaped cross-sectional area of the suture 714. More particularly, the diameter D₂ of the needle 626′ is approximately equal to the major axis P₁ of the suture 714. This formation results in a smaller dragging force during insertion. However, the tissue-holding strength of the suture 714 may be reduced when used with the larger needle 726′ because the tissue was previously cut by the needle 726′ during insertion.

In order to overcome the shortcomings associated with the needles 726 and 726′ illustrated in FIGS. 11A and 11B, respectively, a needle dimensioned to perform well in both insertion and closure phases of stitching is needed. The needle 726″ illustrated in FIG. 11C has an oval-shaped cross-sectional area. The dimensions of the oval-shaped cross-sectional area (i.e., a major axis V₁ and a minor axis V₂) are selected so as to more closely approximate the major axis P₁ and minor axis P₂, respectively, of the parallelogram-shaped cross section of the suture 714. This formation is ideal in that it results in lower dragging force during insertion while maintaining higher tissue-holding strength during wound closure. The preferred average ratio of the dimensions (e.g., cross-sectional area) of the needle 726″ having an oval-shaped cross-sectional area to those of the suture 714 having a parallelogram-shaped cross-sectional area ranges from 0.7 to 1.3.

Unlike the suturing used for other types of tissues, the needle of the present invention defines a large end-to-end radial distance, or “bite,” to enable it to be passed through soft tissues (e.g., fat), and is also strong enough to penetrate tough tissues (e.g., fascia). As the bite diameter increases, the torque acting on the needle tip also increases, which tends to roll, or rotate, the needle away from the surgeon's control and thereby impede the wound suturing procedure. Therefore, a more secure needle-holding structure is required, such as a relatively large needle shaft diameter. However, due to the holding action of the tissue-grasping elements, a small hole in the tissue is preferred. Therefore, a larger needle shaft diameter is not ideal and does not work.

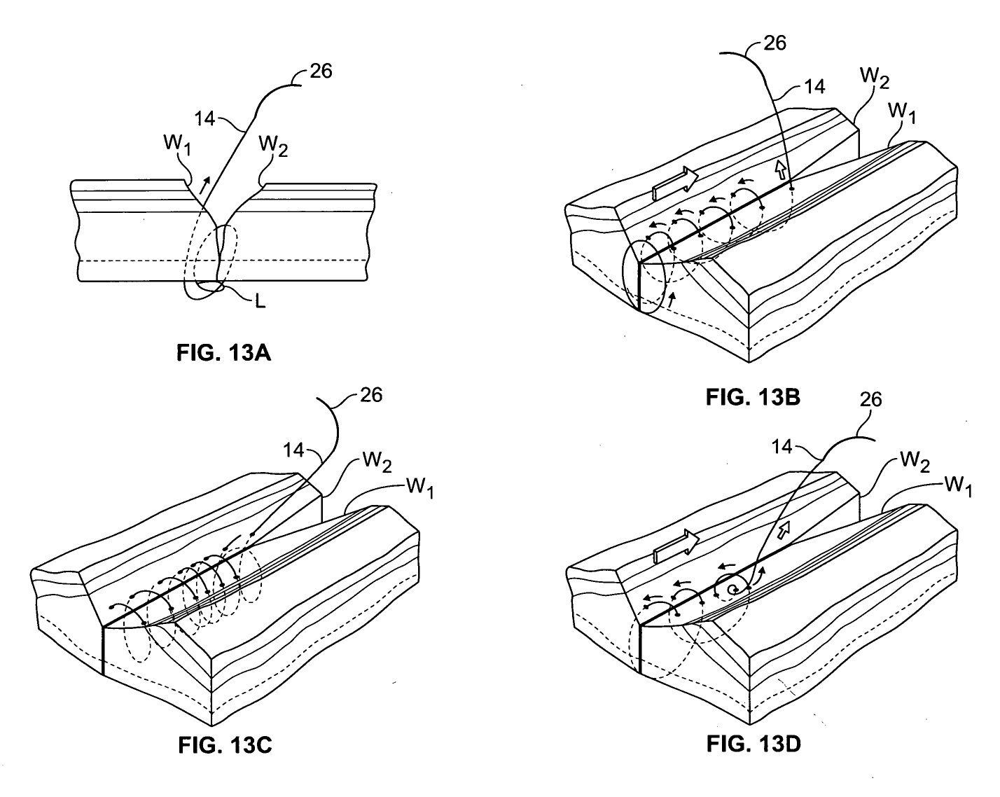

One way to solve this problem involves modifying the needle 26 (shown in FIG. 1) to include a straight portion 54 therein, as illustrated in FIG. 12. The curved portion of the needle 26′ has either a circular cross-sectional area which is shown enlarged in Circle B of FIG. 12, or an oval-shaped cross-sectional area (not shown). In contrast, the straight portion 54 is not curved and has a rectangular cross-sectional area, as shown enlarged in Circle A. The straight portion 54 therefore provides the needle 26′ with a more secure grip, and eliminates the needle rotation due to increased torque described above.

Another way to avoid the increased torque involves the use of a very stiff metal in the manufacture of the needle. Alternatively, a stiffening configuration may be included along the length of the needle shaft.

### Suturing Method

A suturing method is provided for approximating and holding living tissue together for healing using the looped tissue-grasping device according to the present invention and illustrated in FIG. 1. This method offers the advantage of being a completely knotless closure, in contrast to conventional suture closure methods. The method includes the steps of inserting a needle 26 through a first edge of a wound from the underside of the tissue to the top, as shown in FIG. 13A, then drawing the suture 14 through the tissue and keeping the looped end outside of a first edge W₁ of the wound. The needle 26 is then inserted into a second edge W₂ of the wound from top to bottom, exits at the underside of the second edge W₂ and is passed through the suture loop L. The suture 14 is pulled tight to approximate the tissue and lock the first stitch. The tissue-grasping elements are positioned within a specific distance from the apex of the loop or distal end of the suture, so that when the loop end is configured as described and anchors the end of the suture, at least a portion of the number of tissue-grasping elements engage the tissue. To continue the suturing, the needle 26 is inserted into the first edge W₁ from bottom to top so as to be laterally spaced from the previous entry point, exits at the top and is then pushed through the second edge W₂ from top to bottom, so as to be laterally spaced from the previous entry point to complete the second stitch. The suture 14 is pulled to bring the tissues together. The tissue-grasping elements on either side of the loop facing the same direction (not shown) permit movement of the suture 14 through the tissue in the direction of the needle pull and prevent movement of the suture 14 in a direction opposite the movement of the needle 26. As illustrated in FIG. 13B, the steps of the second stitch are repeated to complete the approximation until the wound is completely approximated or until the need to change the suture 14 arises.

Further reference is made to FIG. 13B and also to FIG. 13C. Another suturing method using the looped tissue-grasping device of the present invention is provided for approximating and holding living tissue together for healing. This method includes the steps of inserting the needle 26 through the first edge W₁ of the wound from top to the bottom, then drawing the suture 14 through the tissue and keeping the looped end (not shown) outside of the first edge W₁. The needle 26 is inserted into the second edge W₂ from bottom to top and exits at the top side of the second edge W₂ and passes through the suture loop. The suture 14 is pulled tight to approximate the tissue and lock the first stitch. To continue the suturing, the needle 26 is inserted into the first edge W₁ laterally spaced from the previous entry point and exits at the bottom and is then pushed through the second edge W₂ laterally spaced from the previous entry point to complete the second stitch. The suture 26 is pulled to bring the tissues together. The tissue-grasping elements on either side of the loop facing the same direction (not shown) permit movement of the suture 14 through the tissue in the direction of the needle pull and prevent movement of the suture 14 in a direction opposite the movement of the needle 26. As illustrated in FIG. 13B, the steps of the second stitch are repeated to complete the approximation until the wound is completely approximated or until the need to change the suture 14 arises. When starting the second suture, the first entry point can be immediately adjacent to the last stitch of the first suture or laterally spaced to the last stitch of the first suture. Furthermore, to improve the security of the closure, the first entry point of the second suture can be placed before the last stitch of the first suture, as shown in FIG. 1 3C, to overlap the sutures.

According to the present invention, the loop provides the first stitch anchoring effect to replace the conventional knot. The central clear session length and placement of the tissue-grasping elements near the center region are important factors in determining the security of the anchor.

A suture locking stitch method is provided to enhance the security of the suturing method. When previously-described suturing steps are repeated to a certain number of stitch or at the last stitch, this locking stitch method can be applied, as shown in FIG. 13D. This locking stitch method includes inserting the needle 26 between the last stitch exit point and the exit point before the last stitch exit point on the same side of the tissue and exiting between the insertion point and the last stitch exit point. To complete the locking stitch, the needle 26 is inserted in between the exit point of the locking stitch and the last stitch exit point and exits either before or after the last stitch exit point. The torturous path and the tissue-grasping element engagement in the tissue render a high friction force or resistance for the suture 14 to move, hence strongly holding the tissue together. Experiment results have shown that the performance of this lock stitch is comparable to that of the conventional knot. This lock stitch can be applied intermittently during the suturing to improve the security of the overall closure.

Another suture locking stitch method is provided to enhance the security of the suturing method. This locking stitch method can be applied when the previously-described suturing steps are repeated to a certain number of stitches or at the last stitch. This locking stitch method includes inserting the needle between the last stitch exit point and the exit point before the last stitch exit point on the same side of tissue, and exiting between the insertion point of this locking stitch and the exit point before the last stitch exit point. To complete the locking stitch, the needle is inserted through the exit, along the direction of suturing on the same side of the tissue. The torturous path and the tissue-grasping element engagement in the tissue render a high friction force or resistance for the suture to move, hence strongly holding the tissue together. This lock stitch can be applied intermittently during the suturing to improve the security of the overall closure.

Yet another suture locking stitch method is provided to enhance the security of the suturing method. This locking stitch method can be applied when the previously-described suturing steps are repeated to a certain number of stitches or at the last stitch. This locking stitch method includes inserting the needle between the last stitch exit point and the exit point before the last stitch exit point on the same side of tissue and exiting between the insertion point of this locking stitch and exit point before the last stitch exit point. To complete the locking stitch, the needle is passed under the exposed suture of the locking stitch. The torturous path and the tissue-grasping element engagement in the tissue render a high friction force or resistance for the suture to move, hence strongly holding the tissue together. This lock stitch can be applied intermittently during the suturing to improve the security of the overall closure.

A suturing method using the multiple-looped tissue-grasping device 210 of the present invention (see FIG. 3) is provided for approximating and holding living tissue together or healing. This method includes the steps of inserting the needle through the first edge of the wound from top to bottom, then drawing the suture through the tissue and keeping looped end outside of the first edge. The needle is inserted into the second edge from bottom to top and exits at the top side of the second edge and is passed through the first suture loop. The suture is pulled tight to approximate the tissue and lock the first stitch. To continue the suturing, the needle is inserted into first edge laterally spaced from the previous entry point and exits at the bottom, and is then pushed through the second edge laterally spaced from the previous entry point to complete the second stitch. The suture is pulled to bring the tissues together. The tissue-grasping elements on either side of the loop facing the same direction permit movement of the suture through the tissue in the direction of the needle pull and prevent movement of the suture in a direction opposite the movement of the needle. The steps of the second stitch are repeated to complete the use of the first looped suture or desired length of wound approximation. To continue suturing, the steps of using the first loop are repeated for the second loop, the third loop, and so on until the completion of wound closure or the need to change sutures arises. Furthermore, when finishing the first loop and starting a second loop, the suture can be severed and a second loop may be started as a second suture with multiple loops.

Besides the modifications discussed above, additional modifications can be implemented in the looped tissue-grasping device. For instance, the shape, size and/or construction of the elongated member may be modified. The number, size and/or spatial arrangement of the tissue-grasping elements may also be modified. For example, while the groups of tissue-grasping elements on the two strands are oriented in the same plane as that of the looped suture, as shown in FIGS. 1 and 2, the groups of tissue-grasping elements can alternatively be oriented in a plane perpendicular to the plane of the looped suture and still be parallel to each other. The groups of tissue-grasping elements may also be oriented such that the group on one strand is in a plane perpendicular to the plane containing the group on the other strand.

It should be understood that the embodiments described herein are merely exemplary and that a person skilled in the art may make many variations and modifications thereto without departing from the spirit and scope of the present invention. All such variations and modifications, including those discussed above, are intended to be included within the scope of the invention as defined in the appended claims.

We claim:

1. A tissue-grasping device, comprising a needle and a looped suture attached to said needle, said looped suture having a closed end opposite said needle and first and second strands extending between said closed end and said needle, at least one of said first and second strands having one or more tissue-grasping elements provided thereon.

2. The tissue-grasping device of claim 1, wherein a first plurality of said tissue-grasping elements is provided on said first strand, and a second plurality of said tissue-grasping elements is provided on said second strand.

3. The tissue-grasping device of claim 2, wherein said closed end is not provided with any of said tissue-grasping elements.

4. The tissue-grasping device of claim 3, wherein said first strand includes a first portion proximate said needle, and said second strand includes a first portion proximate said needle, both of said first portions being void of any of said tissue-grasping elements.

5. The tissue-grasping device of claim 4, wherein said first strand includes a second portion proximate said closed end, and said second strand includes a second portion proximate said closed end, both of said second portions being void of any of said tissue-grasping elements.

6. The tissue-grasping device of claim 2, wherein said first plurality of tissue-grasping elements is aligned with said second plurality of tissue-grasping elements, such that each of said tissue-grasping elements on said first strand is aligned, in a lateral direction, with a corresponding one of said tissue-grasping elements on said second strand.

7. The tissue-grasping device of claim 2, wherein said first plurality of tissue-grasping elements is not aligned with said second plurality of tissue-grasping elements, such that each of said tissue-grasping elements on said first strand is offset, in a lateral direction, from a corresponding one of said tissue-grasping elements on said second strand.

8. The tissue-grasping device of claim 2, wherein said first strand includes an inner lateral surface proximate to said second strand and an outer lateral surface distal to said second strand, and said second strand includes an inner lateral surface proximate said first strand and an outer lateral surface distal to said first strand, at least one of said inner and outer lateral surfaces of said first strand being provided with said first plurality of said tissue-grasping elements and at least one of said inner and outer lateral surfaces of said second strand being provided with said second plurality of said tissue-grasping elements.

9. The tissue-grasping device of claim 8, wherein said outer lateral surface of said first strand is provided with said first plurality of said tissue-grasping elements, and said outer lateral surface of said second strand is provided with said second plurality of said tissue-grasping elements.

10. The tissue-grasping device of claim 8, wherein said inner lateral surface of said first strand is provided with said first plurality of said tissue-grasping elements, and said inner lateral surface of said second strand is provided with said second plurality of said tissue-grasping elements.

11. The tissue-grasping device of claim 8, wherein some of said first plurality of said tissue-grasping elements are provided on said inner lateral surface of said first strand and some of said first plurality of said tissue-grasping elements are provided on said outer lateral surface of said first strand, and wherein some of said second plurality of said tissue-grasping elements are provided on said inner lateral surface of said second strand and some of said second plurality of said tissue-grasping elements are provided on said outer lateral surface of said second strand.

12. The tissue-grasping device of claim 2, wherein each of said tissue-grasping elements of said first and second pluralities of said tissue-grasping elements includes a leading edge proximate to said needle and a trailing edge distal to said needle, said leading and trailing edges having a concaved shape where they merge with said looped suture.

13. The tissue-grasping device of claim 12, wherein said concaved shape of said trailing edge forms a recess which extends laterally into said looped suture.

14. The tissue-grasping device of claim 12, wherein said tissue-grasping elements are substantially shark fin-shaped.

15. The tissue-grasping device of claim 12, wherein said trailing edge includes a plurality of serrations thereon.

16. The tissue-grasping device of claim 8, wherein said inner lateral surfaces of said first and second strands are substantially flat.

17. The tissue-grasping device of claim 16, wherein each of said first and second strands has a triangular-shaped cross section such that each of said strands includes three apexes and three sides, said tissue-grasping elements of said first strand being provided on the apex which defines said outer lateral surface of said first strand and said tissue-grasping elements of said second strand being provided on the apex which defines said outer lateral surface of said second strand.

18. The tissue-grasping device of claim 17, wherein, when said inner lateral surfaces of said first and second strands are juxtaposed, said first and second strands create a combined cross-sectional shape which is a parallelogram having a minor axis and a major axis.

19. The tissue-grasping device of claim 18, wherein said needle has a cross-sectional shape which is an oval having a minor axis, which is substantially the same length as the minor axis of said parallelogram, and a major axis, which is substantially the same length as the major axis of said parallelogram.

20. The tissue-grasping device of claim 1, further comprising at least one supplemental strand of suture extending between said first and second strands so as to form at least one additional closed loop.

21. The tissue-grasping device of claim 1, wherein said first strand is not provided with any of said tissue-grasping elements.

22. The tissue-grasping device of claim 1, wherein said needle includes a curved portion, which has a substantially circular cross-sectional shape, and a substantially straight portion, which has a substantially rectangular cross-sectional shape.

23. The tissue-grasping device of claim 1, wherein said needle includes a curved portion, which has a substantially oval cross-sectional shape, and a substantially straight portion, which has a substantially rectangular cross-sectional shape.

24. The tissue-grasping device of claim 1, wherein said needle has a first cross-sectional shape and said suture has a second cross-sectional shape, said second cross-sectional shape being substantially similar to said first cross-sectional shape.
